(19) United States
(12) Patent Application Publication
(10) Pub. No.: US 2017/0332796 A1
(43) Pub. Date: Nov. 23, 2017

(54) BACK SUPPORT DEVICES WITH INTEGRATED THERMAL THERAPY AND METHODS OF USING THE SAME

(71) Applicant: Katherine Krug, Las Vegas, NV (US)

(72) Inventor: Katherine Krug, Las Vegas, NV (US)

(21) Appl. No.: 15/161,146

(22) Filed: May 20, 2016

(51) Int. Cl. A47C 16/00; A61H 11/00; A61F 5/37; A61H 1/00; A61F 7/08; A61F 7/00

(52) U.S. Cl. CPC A47C 16/005; A61H 1/006; A61F 7/08; A61H 11/00; A61F 5/37; A61F 2007/0027; A61H 2201/02

(57) ABSTRACT

An integrated storage pouch apparatus for a back support and thermal therapy device for supporting and applying thermal therapy to a user's back which includes a back support member having a first surface and an opposing second surface, the first surface configured to face and support a user's back, a thermal therapy storage compartment integrally formed between the first and second surfaces of the back support member and configured to store a thermal pack, first and second adjustable strap members coupled to opposite ends of the back support member, each of the first and second adjustable strap members are configured to wrap around a user's knee, wherein the back support member folds inside out to through the thermal therapy storage compartment to form a strap storage compartment configured to receive and store the first and second adjustable strap members. The combined back support and thermal therapy apparatus provides thermal therapy to the user, while at the same time using the user's knees to help support the user in a proper healthy posture, when sitting.

### BACKGROUND OF THE INVENTION

### 1. Field of the Invention

The present general inventive concept relates to back support devices, systems, and methods, and more particularly to back support devices configured to support a user in a proper posture while seated and also to provide thermal therapy to the user's back and/or knees. In addition, the back support devices according to the present general inventive concept may further include integrated compartments to store thermal packs to apply thermal therapy to the user's back and/or knees.

### 2. Description of Related Art

Back support devices which help users maintain a healthy posture have been previously developed. A person's posture refers to his or her body alignment and position with respect to gravity while standing, sitting, or lying down. Proper posture decreases an amount of stress on ligaments holding the person's joints and spine together and reduces fatigue since muscles are being used more efficiently, and therefore allows the person's body to use less energy.

According to many medical professionals, the proper healthy posture for sitting in a chair requires one to sit up with his or her back straight and shoulders back. The person's knees should be maintained at a right angle and even with or slightly higher than his or her hips. In addition, the person's feet should be kept flat on the floor to distribute the user's body weight evenly on both hips. However, many people are unaware of this proper sitting posture and therefore often experience neck and back pain and fatigue. As a result, several back support devices have been developed which help support users to sit in proper healthy postures. Thermal therapy has been shown to have several beneficial therapeutic effects when used for rehabilitation purposes. However, conventional back support devices fail to provide thermal therapy to a user's back and/or knees.

As such, there remains a need for back support devices which help support a users in a proper posture, while at the same time applying thermal therapy to the user's back and/or knees.

### SUMMARY OF THE INVENTION

The present general inventive concept provides a combined back support and thermal therapy apparatus that provides thermal therapy to the user, while at the same time using the user's knees to help support the user in a proper healthy posture, when sitting.

The foregoing and/or other aspects of the present general inventive concept may be achieved by providing a combined back support and thermal therapy apparatus for supporting and applying thermal therapy to a user's back which includes a back support member having a first surface and an opposing second surface, the first surface configured to face and support a user's back, a thermal therapy storage compartment integrally formed between the first and second surfaces of the back support member and configured to store a thermal pack, first and second adjustable strap members coupled to opposite ends of the back support member, each of the first and second adjustable strap members are configured to wrap around a user's knee, wherein the back support member folds along a center region to form a strap storage compartment defined within the second surface, the strap storage compartment configured to receive and store the first and second adjustable strap members.

The combined back support and thermal therapy apparatus may further include a tension application member coupled to one of the first adjustable strap member and the second adjustable strap member.

The tension application member may be configured to allow the user to adjust and fix a length of the adjustable strap member.

The user's knees may apply a force to the back support member through first and second adjustable straps to support the user in a proper posture position.

The combined back support and thermal therapy apparatus may further include knee pads coupled to the first and second adjustable strap members, the knee pads configured to distribute a force over a surface of the user's knees.

The combined back support and thermal therapy apparatus may further include a thermal therapy storage compartment integrally formed with the knee pads and configured to store a thermal pack to provide thermal therapy to the user's knees.

The knee pads may be formed in a wedge shape to evenly distribute a force over a surface of the user's knees.

The combined back support and thermal therapy apparatus may further include a sleeve formed on the first surface of the back support member configured to provide access to the thermal therapy storage compartment.

The combined back support and thermal therapy apparatus may further include a fastening member coupled to a perimeter of the back support member to open and close the strap storage compartment from an external environment.

The fastening member may include one of a snap, a button, a zipper, a velcro™ and a clip.

The combined back support and thermal therapy apparatus may further include a clip member configured to detachably connect the first adjustable strap member to the second adjustable strap member to prevent the user's knees from spreading apart.

The foregoing and/or other aspects of the present general inventive concept may be also achieved by providing an integrated storage pouch apparatus for a back support and thermal therapy device for supporting and applying thermal therapy to a user's back. The apparatus includes a back support member having a first surface and an opposing second surface, the first surface configured to face and support a user's back, a thermal therapy storage compartment integrally formed between the first and second surfaces of the back support member and configured to store a thermal pack, first and second adjustable strap members coupled to opposite ends of the back support member, each of the first and second adjustable strap members are configured to wrap around a user's knee, wherein the back support member folds inside out to through the thermal therapy storage compartment to form a strap storage compartment configured to receive and store the first and second adjustable strap members.

The integrated storage pouch apparatus may further include a tension application member coupled to one of the first adjustable strap member and the second adjustable strap member to adjust a length of the adjustable strap member.

The user's knees may apply a force to the back support member through first and second adjustable straps to support the user in a proper posture position.

The integrated storage pouch apparatus may further include knee pads coupled to the first and second adjustable strap members, the knee pads configured to distribute a force over a surface of the user's knees.

The knee pads may be formed in a wedge shape to evenly distribute a force over a surface of the user's knees.

The integrated storage pouch apparatus may further include a thermal therapy storage compartment integrally formed with the knee pads and configured to store a thermal pack to provide thermal therapy to the user's knees.

The integrated storage pouch apparatus may further include a fastening member coupled to a top end of the back support member to open and close the thermal therapy storage compartment from an external environment.

The fastening member may be configured to open and close the thermal therapy storage compartment and the strap storage compartment from an external environment when the back support member is flipped inside out.

The integrated storage pouch apparatus may further include a clip member configured to detachably connect the first adjustable strap member to the second adjustable strap member to prevent the user's knees from spreading apart.

Additional aspects of the present general inventive concept will be set forth in part in the description which follows and, in part, will be obvious from the description, or may be learned by practice of the general inventive concept.

### BRIEF DESCRIPTIONS OF THE DRAWINGS

These and/or other aspects of the present general inventive concept will become apparent and more readily appreciated from the following description of the various embodiments, taken in conjunction with the accompanying drawings of which:

FIG. 1 illustrates a back view of a back support device having an integrated thermal therapy compartment according to an example embodiment of the present general inventive concept;

FIG. 2 illustrates a front view of the back support device illustrated in FIG. 1;

FIG. 3 illustrates a perspective view of a user using the back support device illustrated in FIG. 1;

FIG. 4 illustrates a side view of the user using the back support device illustrated in FIG. 3;

FIGS. 5A-B illustrate knee pads according to example embodiments of the present general inventive concept;

FIG. 6 shows a perspective view of the back support device illustrated in FIG. 1 in a partially-folded state showing a strap storage compartment used to store adjustable strap members;

FIG. 7 illustrates a perspective view of a user using a back support device according to another example embodiment of the present general inventive concept;

FIGS. 8A-8C illustrate a back support device and method of storing the adjustable strap members within the back support device according to another example embodiment of the present general inventive concept;

FIGS. 9A-9D illustrate a back support device and method of storing the adjustable strap members within the back support device according to another example embodiment of the present general inventive concept;

FIG. 10 illustrates a front view of a back support device having an integrated thermal therapy storage compartment according to another example embodiment of the present general inventive concept;

FIG. 11A-11B illustrates a thermal pack and a front view of a back support device according to another example embodiment of the present general inventive concept;

FIG. 12 illustrates a wrap-around thermal pack according to an example embodiment of the present general inventive concept;

FIGS. 13A-13E illustrate a back support device and method of storing the adjustable strap members in the back support device according to another example embodiment of the present general inventive concept; and

FIG. 14 illustrates a back support device according to another example embodiment of the present general inventive concept.

### DETAILED DESCRIPTION

Reference will now be made in detail to the exemplary embodiments of the present general inventive concept, examples of which are illustrated in the accompanying drawings, wherein like reference numerals refer to the like elements throughout. The exemplary embodiments are described below in order to explain the present general inventive concept by referring to the figures.

The present general inventive concept relates to back support devices, systems, and methods, and more particularly to back support devices which are configured to hold a thermal pack (e.g., an ice pack or a heating pad) adjacent to a user's back and/or knees to provide thermal therapy to the user, while at the same time using the user's knees to help support the user in a proper healthy posture when sitting.

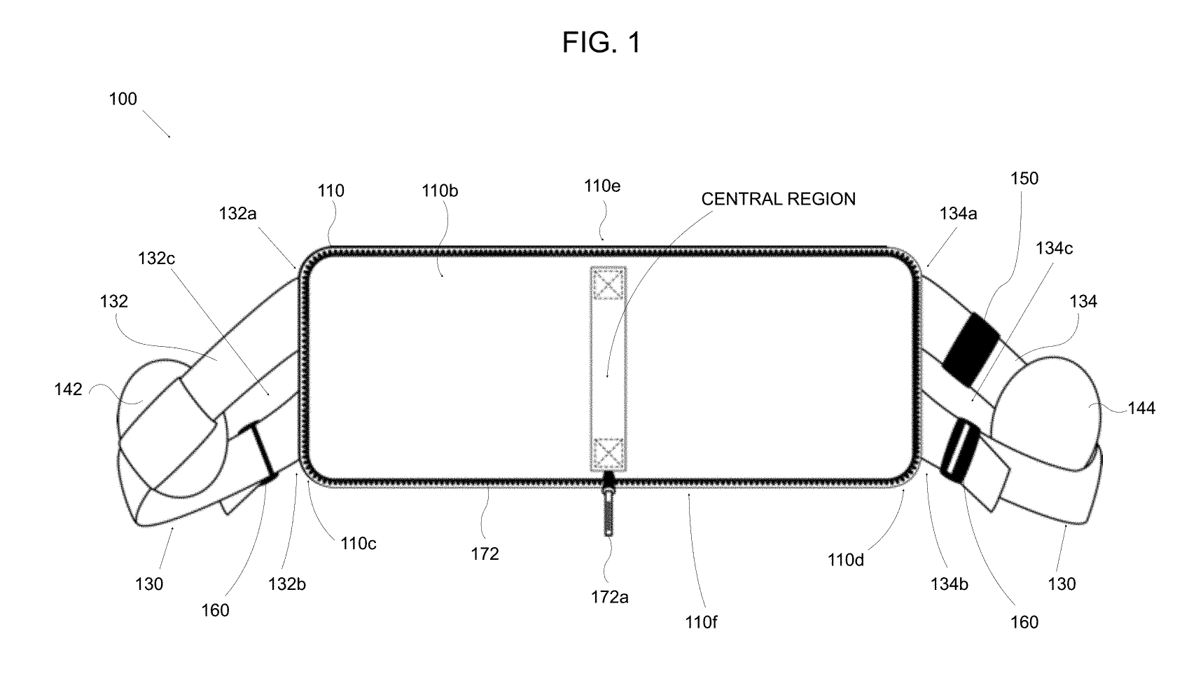

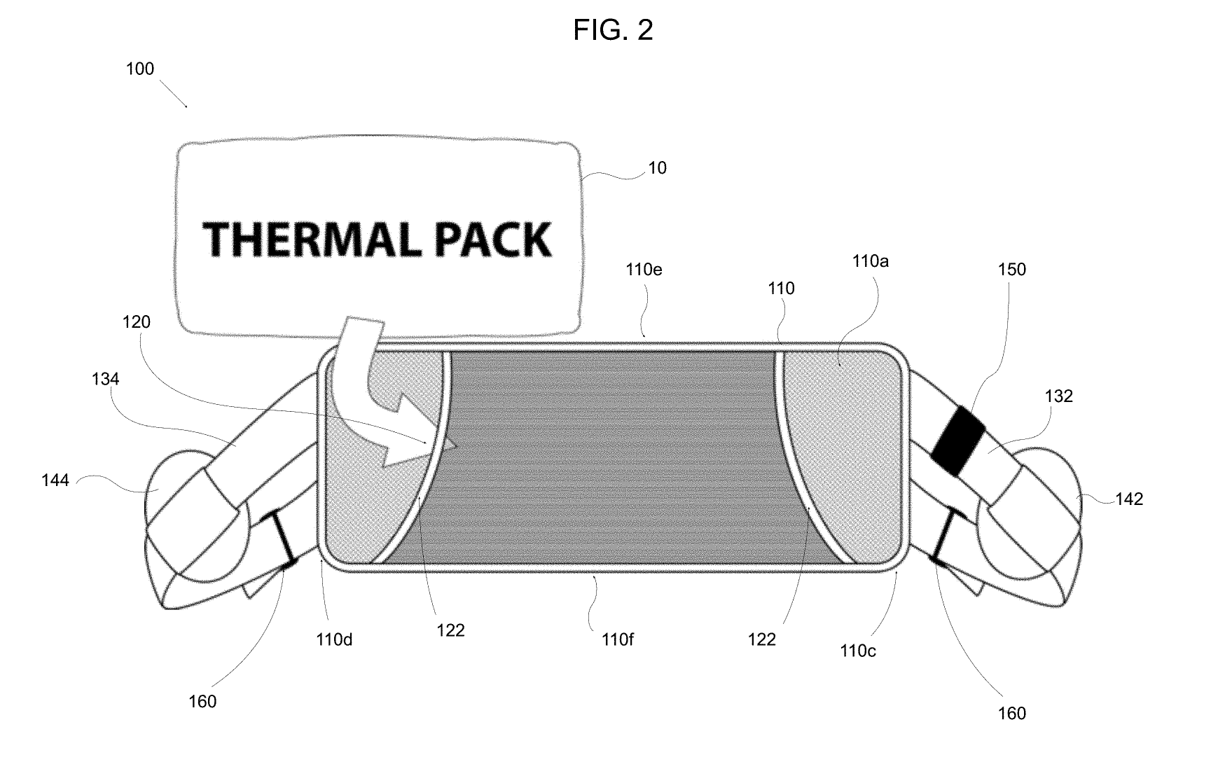

FIG. 1 illustrates a back view of a back support device 100 having an integrated thermal therapy compartment 120 according to an example embodiment of the present general inventive concept and FIG. 2 illustrates a front view of the back support device 100 illustrated in FIG. 1.

Referring to FIGS. 1 and 2, the back support device 100 according to the present general inventive concept includes a back support member 110 having a first surface 110a (i.e., a front surface) and a second surface 110b (i.e., an opposing back surface), an integrated thermal pack compartment 120 formed on the front surface 110a of the back support member 110 to store a thermal pack 10 (e.g., an ice pack and/or a heating pad), and a plurality of adjustable strap members 130 attached to the back support member 110. In use, the front surface 110a may be placed adjacent to and/or against the user's back 20 to simultaneously provide back support and thermal therapy to the user 20. The back support member 110 may be configured to receive and store the plurality of adjustable strap members 130.

In the present example embodiment, the back support member 110 may be formed in a rectangular shape having a first end 110c, a second end 110d, a top end 110e, and a bottom end 110f. The back support member 110 may include a padding material disposed in between the front surface 110a and the back surface 110b to provide support, thermal therapy, and/or comfort to a user's back 20a. The back support member 110 is configured to engage and support a region of the user's back 20a (e.g., lumbar). The padding material may be formed of foam, cotton, or the like. However, the present general inventive concept is not limited thereto. That is, in alternative example embodiments, the back support member 110 may be formed in various shapes and/or sizes and may be manufactured from various types of materials. In addition, the padding material may include a thermal absorbing material which may absorb heat and/or cold to further apply thermal therapy to the user 20.

Referring now to FIG. 2, the back support device 100 may further include an integrated thermal therapy storage compartment 120 formed on the front surface 110a of the back support member 110. The thermal therapy storage compartment 120 is configured to store a thermal pack 10 and to support the thermal pack 10 against or adjacent to the user's back to provide thermal therapy to the user 20. The thermal pack 10 may provide cold and/or heat therapy to the user. However, the present general inventive concept is not limited thereto. The thermal pack 10 may further include a means for generating a vibration to provide massage therapy to the user and/or various other therapies including magnetic field or other metallic type therapies, such as copper.

In the present example embodiment, the thermal therapy storage compartment 120 may be formed as a pocket or sleeve 122 which may be formed on the front surface 110a of the back support member 110. The sleeve 122 may be formed from an elastic material to support and securely hold the thermal pack 10 within the thermal therapy storage compartment 120.

Referring to FIGS. 1-2, in the present example embodiment, the back support device 100 includes a first adjustable strap member 132 and a second adjustable strap member 134 coupled to opposite ends of the back support member 110. The first adjustable strap member 132 includes a first end 132a and an opposite second end 132b which are both coupled to the first end 110c of the back support member 110 to thereby form a first loop 132c. Similarly, the second adjustable strap member 134 includes a first end 134a and an opposite second end 134b which are coupled to the second end 110d of the back support member 110 to thereby form a second loop 134c.

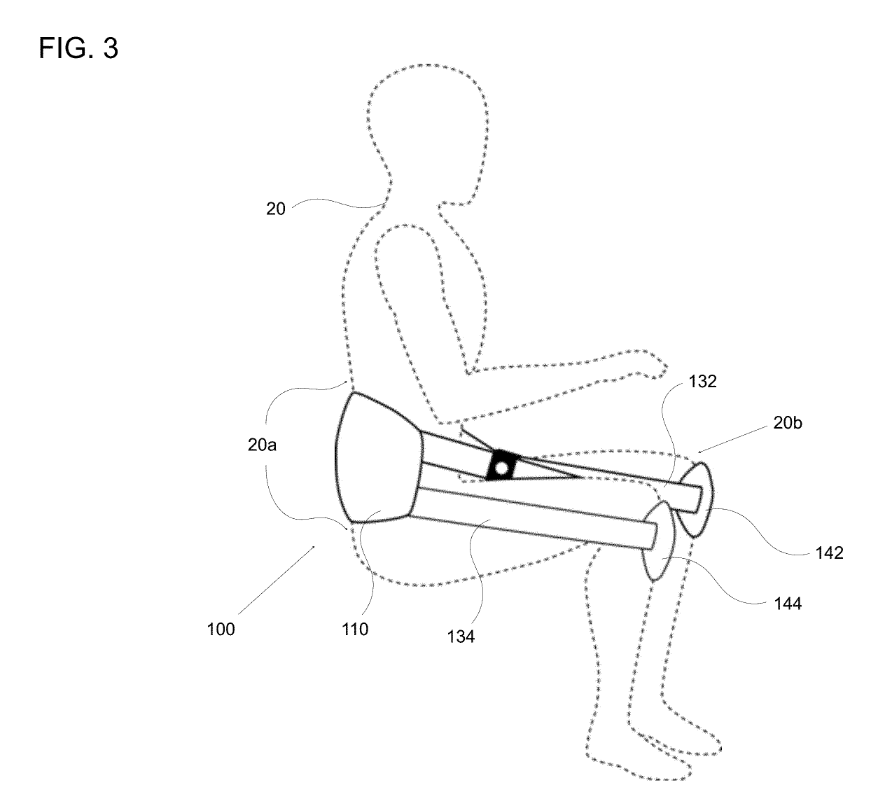

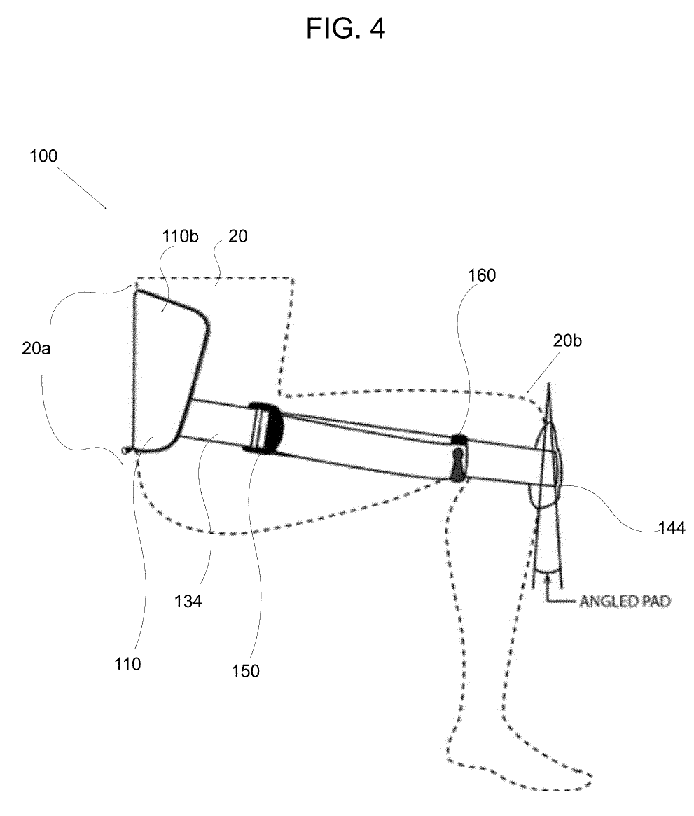

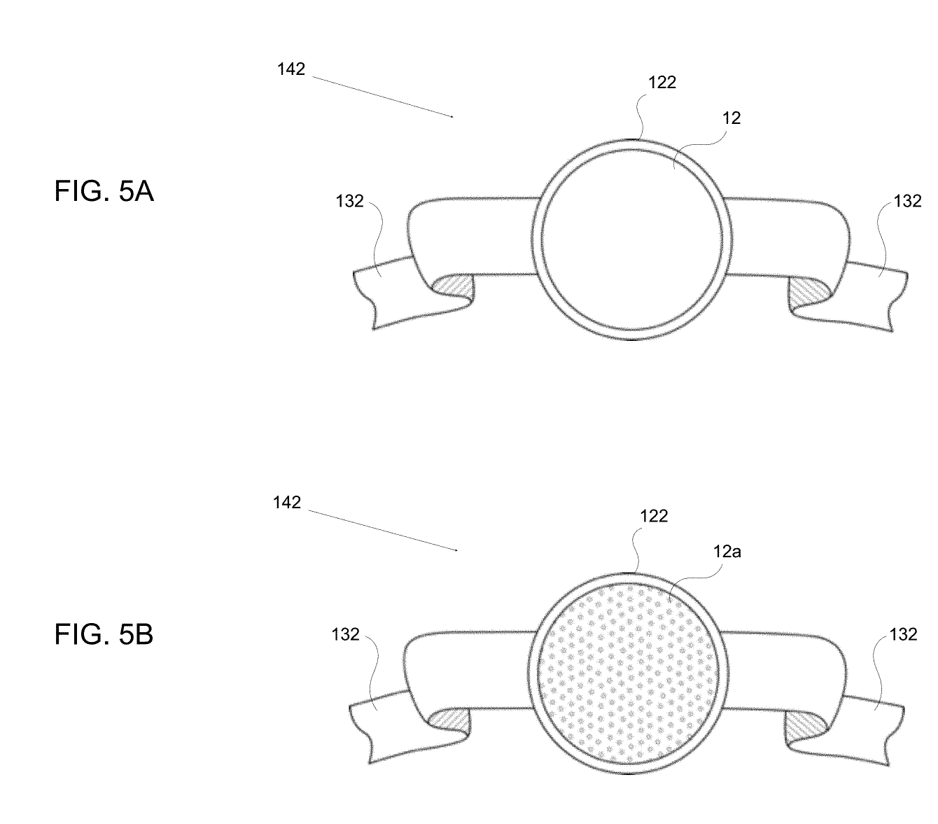

FIG. 3 illustrates a perspective view of a user 20 using the back support device 100 illustrated in FIG. 1 and FIG. 4 illustrates a side view of the user 20 using the back support device 100 illustrated in FIG. 3. FIGS. 5A-B illustrate knee pads according to example embodiments of the present general inventive concept.

Referring to FIGS. 3 and 4, the first and second loops 132c and 134c of the first and second adjustable strap members 132 and 134, respectively, are configured to wrap around a user's knees 20b while the user 20 remains in a seated position in order to support the user's back 20a. The first and second loops 132c and 134c are sized, shaped, and formed so as to easily slide over the user's knees 20b, when the user 20 is in a seated position.

In alternative example embodiments, one of the first or second adjustable strap members 132 and 134 may be manufactured at a fixed length and the other adjustable strap member 132 or 134 may be configured to be adjustable by a user 20 in order to control an amount of force applied by the back support member 110 on the user's back 20a. That is, the first and/or second adjustable strap member 132 and 134 may be manufactured from an elastic material so as to provide adequate tension on the back support member 110. However, the present general inventive concept is not limited thereto.

The back support device 100 is configured to assist the user 20 to achieve and maintain a proper healthy posture while sitting and also to simultaneously provide thermal therapy to the user's back 20a and/or knees 20b.

The back support member 110 is first placed around the user's back 20a such that the first surface 110a (i.e., front surface) of the back support member 110 faces the user's back 20a. As such, the thermal pack 10 stored within the integrated thermal storage compartment 120 formed on the first surface 110a of the back support member 110 may directly or indirectly contact the user's back 20. In use, the back support member 110 may simultaneously provide back support and thermal therapy to the user's back 20a.

Next, the first and second loops 132c and 134c of the first and second adjustable strap members 132 and 134 may then wrapped around the user's knees 20b. The user may then adjust the first and second adjustable strap members 132 and 134 using a clip member 150 and a tension application member 160 coupled to the first and second adjustable strap members 132 and 134 so that the back support member 110 is pressed firmly against the user's 20 back.

Referring to FIG. 4, the back support device 100 includes a back support member 110 coupled to first and second adjustable strap members 132 and 134 which are wrapped or looped around the user's 20 knees to help assist the user 20 to maintain in a proper posture, while sitting. In particular, the back support device 100 utilizes the users knees to provide support to the user's back. The tension created by adjusting the first and second adjustable strap members 132 and 134 helps maintain the user's knees at a right angle and even with the user's hips.

In the present example embodiment, the back support device 100 further includes a clip member 150 and a tension application member 160 coupled to the first and second adjustable strap members 132 and 134. The clip member 150 allows the user to connect the first adjustable strap member 132 to the second adjustable strap member 134 to thereby prevent the user's knees from splaying (i.e., spreading) too far apart. The tension application member 160 allows the user to adjust a length of the adjustable strap. As a result, the user's knees helps support the back support member 110 against the user's back such that the user 20 is held in a proper healthy posture, while sitting, and therefore experiences reduced stress and/or fatigue.

The tension member 160 may allow the adjustable strap member to move in a first direction to tighten the strap member against the user's knees and prevents the strap from moving in the opposite direction in order to fix a length of the first and second adjustable strap members 132, 134 as adjusted and set by the user.

Referring now to FIG. 3, in alternative example embodiments of the present general inventive concept, the back support device 100 may further include first and second knee pads 142 and 144 which are movably coupled on the first and second adjustable strap members 132 and 134, respectively. That is, a position of the first and second knee pads 142 and 144 may be adjusted by a user along a length of the first and second adjustable strap members 132 and 134 to accommodate various sized users.

In the present example embodiment, the first knee pad 142 may be positioned within a first loop 132c formed with the first adjustable strap member 132, and the second knee pad 144 may be positioned within a second loop 134c formed with the second adjustable strap member 134. The first and second knee pads 142, 144 are configured to correspond to a size and/or shape of the user's knees 20b. However, the present general inventive concept is not limited thereto.

Referring to FIG. 4, in the present example embodiment, the first and second knee pads 142 and 144 are formed in a wedge-like or angular shape in order to evenly spread a force generated by the adjustable straps 132 and 134 over an area or surface of the user's knees 20b to minimize or reduce an amount of stress and/or fatigue to the user's knees. However, the present general inventive concept is not limited thereto. That is, in alternative example embodiments, the first and second knee pads 142 and 144 may be formed in various shapes and/or sizes in order to accommodate various sized users.

In use, the front surface 110a of the back support member 110 may be placed adjacent to or against the user's 20 back. The first loop 132c of the first adjustable strap member 132 may be wrapped around the user's left knee, and the second loop 132c of the second adjustable strap member 134 may then be wrapped around the user's right knee. The first knee pad 142 may be positioned over the user's right knee and the second knee pad 144 may be positioned over the user's left knee. The user may then adjust the first and second adjustable strap members 132, 134 so that the back support member 110 is firmly pressed against the user's 20 back to support the user in a proper healthy upright posture, while sitting. In addition, the thermal pack 10 which is stored within the thermal therapy storage compartment 120 may be pressed against the user's 20 back to thereby apply thermal therapy to the user 20.

Referring to FIG. 5A, in alternative example embodiments, the first and second knee pads 142 and 144 may further include a thermal therapy storage compartment 122 integrally formed with the knee pads and configured to store a thermal pack 12 to provide thermal therapy to the user's knees 20b.

Referring to FIG. 5B, in alternative example embodiments, the first and second knee pads 142 and 144 may further include a thermal therapy storage compartment 122 integrally formed with the knee pads and configured to store a thermal pack 12a having a textured surface thereon to provide thermal and/or acupressure therapy to the user's knees 20b. That is, in the present example embodiment, the textured surface may include protrusions and/or knobs extending from the knees pads to provide massage therapy to the user's knees 20b.

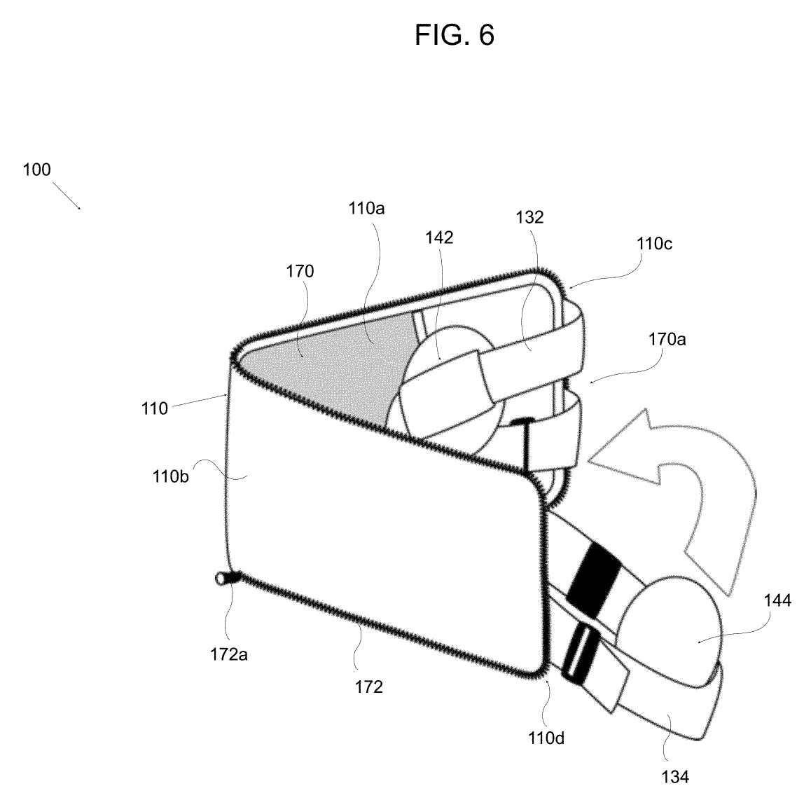

FIG. 6 shows a perspective view of the back support device 100 illustrated in FIG. 1 in a partially-folded state showing a strap storage compartment 170 used to store the adjustable strap members 132 and 134.

Referring to FIG. 6, the back support device 100 may further include a strap storage compartment 170 formed in between the front and back surfaces 110a and 110b of the back support member 110. The strap storage compartment 170 is configured to store the first and second adjustable strap members 132 and 134. In the present example embodiment, the strap storage compartment 170 may be formed when the back support member 110 is folded in half about a central region. That is, when the first end 110c of the back support member 110 is folded toward the second end 110d a cavity is formed which may be sealed off from an external environment with a fastening member 172 having a zipper member 172a used to open and close the strap storage compartment 170.

In the present example embodiment, the first adjustable strap member 132 and the second adjustable member 134 may be inserted into a first opening 170a in order to be stored within the strap storage compartment 170. The back support member 110 may further include a fastening member 172, such as a zipper or velcro member, which is configured to enclose the first and second adjustable strap members 132 and 134 within the strap storage compartment 170. The fastening member 172 may be coupled to an outer perimeter of the back support member 110 and may include a zipper 172a or the like to open and close the strap storage compartment 170.

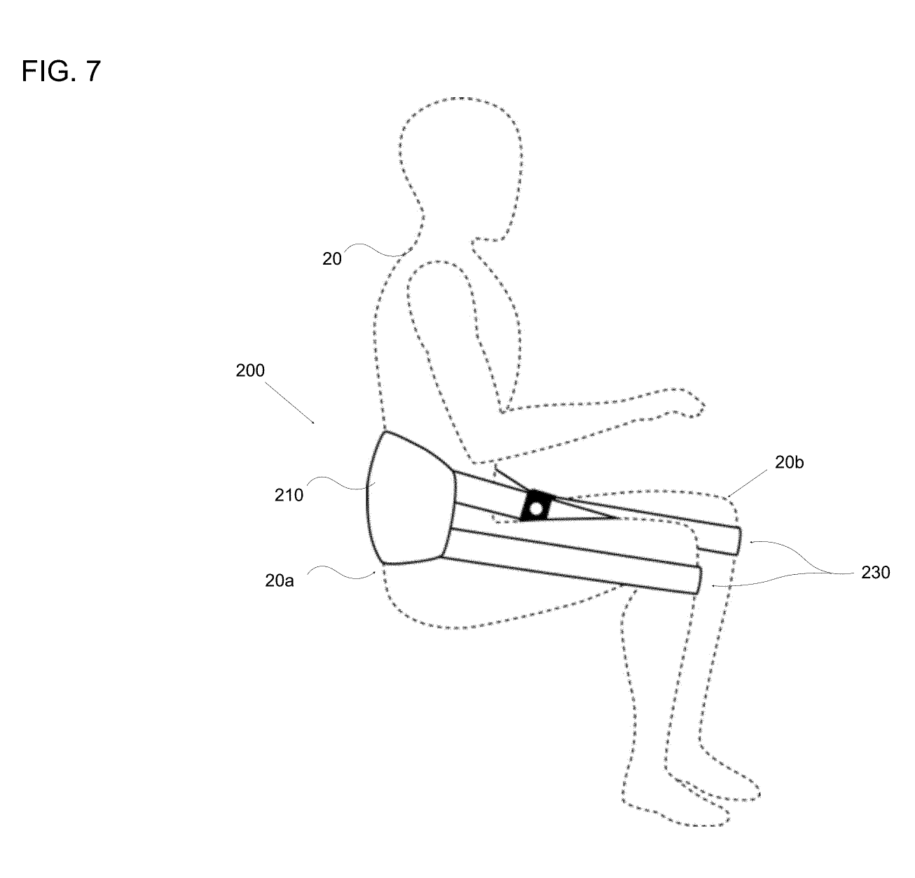

FIG. 7 illustrates a perspective view of a user using a back support device 200 according to another example embodiment of the present general inventive concept.

Referring FIG. 7, the back support device 200 according to the present embodiment is similar to the previous example embodiment. However, the back support device 200 of the present example embodiment may also function to simultaneously provide back support and thermal therapy to the user 20, without requiring the use of knee pads as described above.

In the present example embodiment, the back support device 200 includes a back support member 210, an integrated thermal pack storage compartment formed within the back support member 210 and a plurality of adjustable strap members 230 attached to the back support member 210 to help support the user in a proper healthy posture, while sitting. Although not illustrated, the thermal pack storage compartment is integrally formed within the back support member 210 with a non-removable thermal pack integrated therein. The entire back support device 200 may be manufactured from a thermal absorbing material which may absorb heat and/or cold. As such, the back support device 200 according to the present example embodiment may be placed in a microwave or freezer to provide thermal therapy to the user.

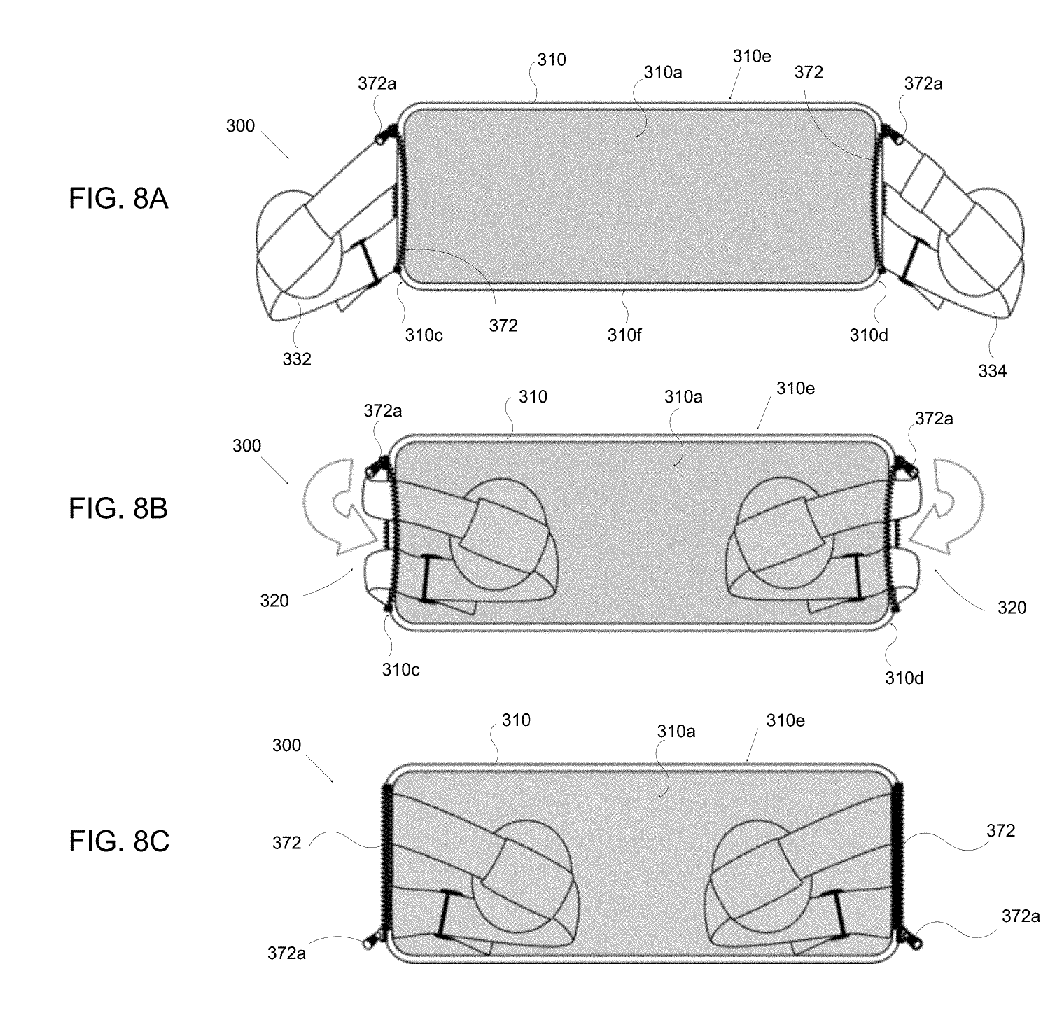

FIGS. 8A-8C illustrate a back support device 300 and method of storing the adjustable strap members 332 and 334 within the back support device 300 according to another example embodiment of the present general inventive concept.

Referring to FIGS. 8A-8C, the back support device 300 according to the present general inventive concept includes a back support member 310 having a first surface 310a (i.e., a front surface) and a second surface 310b (i.e., an opposing back surface), an integrated thermal pack compartment 320 formed on the front surface 310a of the back support member 310 to store a thermal pack 10 (e.g., an ice pack and/or a heating pad), and first and second adjustable strap members 332 and 334 attached to the back support member 110. In use, the front surface 310a may be placed adjacent to and/or against the user's back 20 to simultaneously provide back support and thermal therapy to the user 20. The back support member 310 may be configured to receive and store the plurality of adjustable strap members 332 and 334.

In the present example embodiment, the back support member 310 may be formed in a rectangular shape having a first end 310c, a second end 310d, a top end 310e, and a bottom end 310f. The back support member 310 may include a padding material disposed in between the front surface 310a and the back surface 310b to provide support, thermal therapy, and/or comfort to a user's back 20a. The back support member 310 is configured to engage and support a region of the user's back 20a (e.g., lumbar). The padding material may be formed of foam, cotton, or the like. However, the present general inventive concept is not limited thereto. That is, in alternative example embodiments, the back support member 310 may be formed in various shapes and/or sizes and may be manufactured from various types of materials. In addition, the padding material may include a thermal absorbing material which may absorb heat and/or cold to further apply thermal therapy to the user.

The first and second adjustable strap members 332 and 334 may be coupled to the first and second ends 310c and 310d of the back support member 310. In the present example embodiment, the first and second adjustable strap members 332 and 334 may be inserted into and stored within the integrated thermal pack compartment 320 formed in the back support member 310. In addition, the back support member 310 may further include a fastening member 372 having a zipper member 372a used to open and close both ends of the back support member 310 in order to secure the first and second adjustable strap members 332 and 334 within the thermal pack compartment 320.

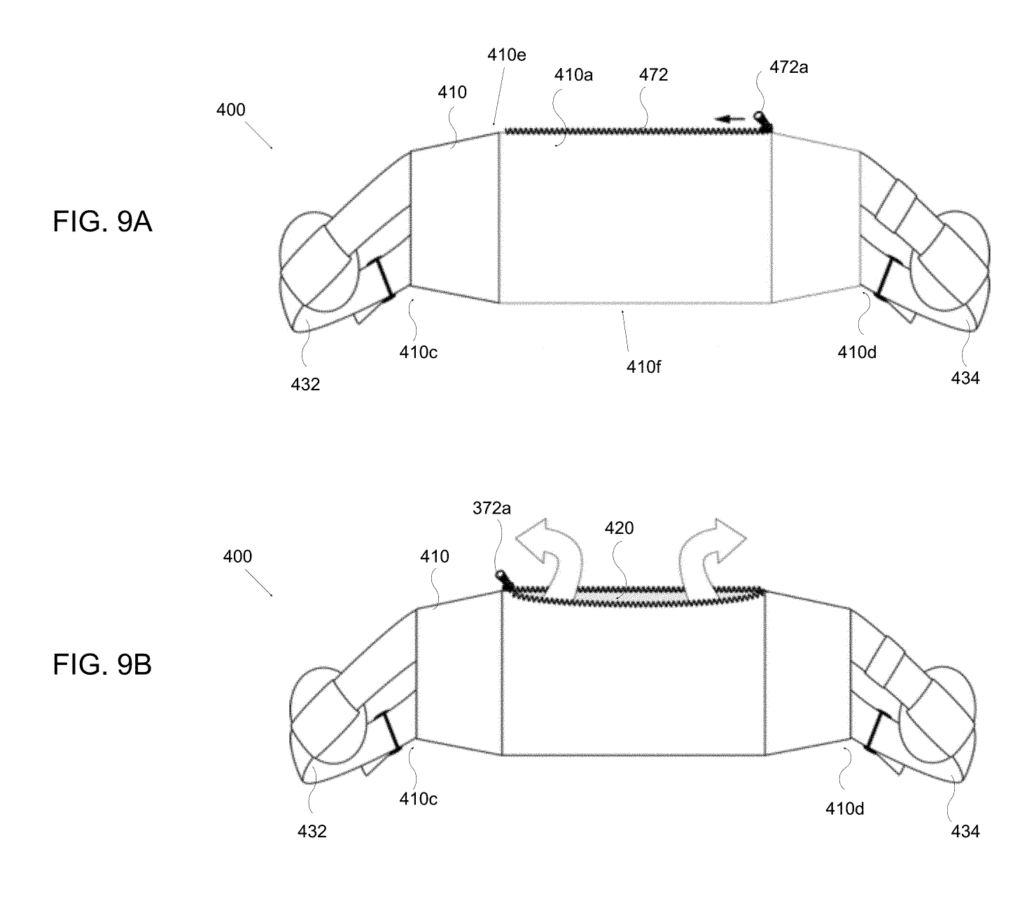

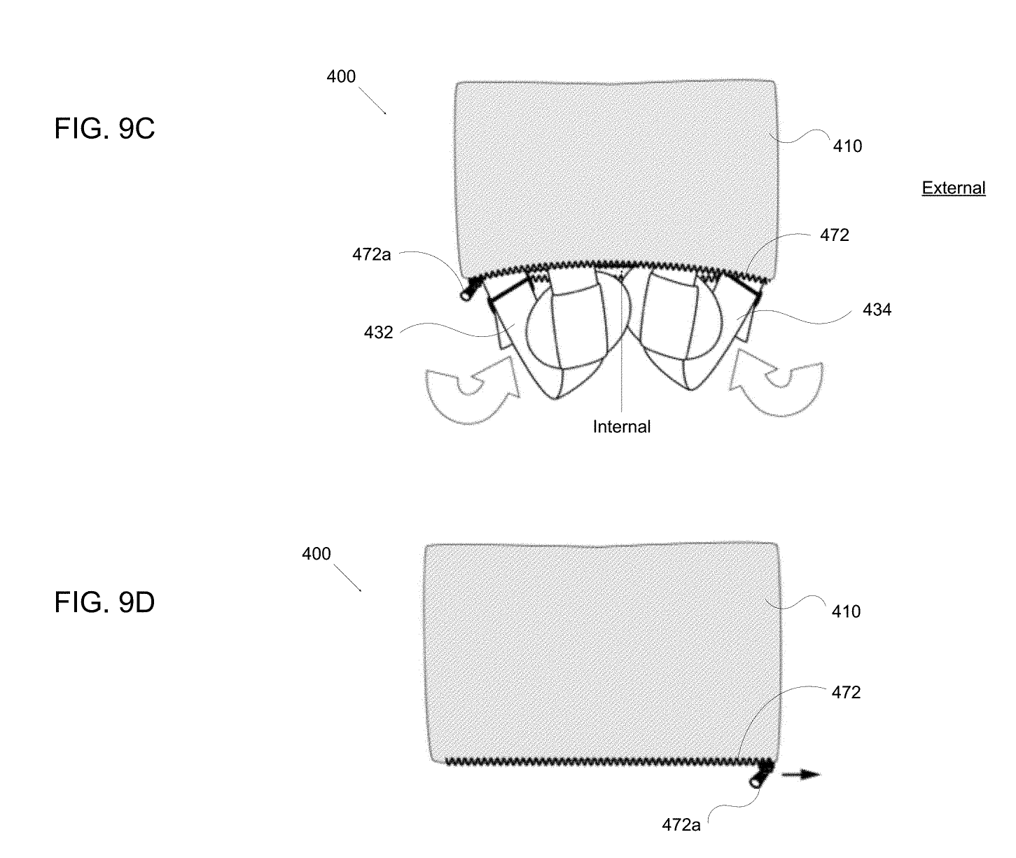

FIGS. 9A-9D illustrate a back support device 400 and method of storing the adjustable strap members within the back support device 400 according to another example embodiment of the present general inventive concept.

Referring to FIGS. 9A-9D, the back support device 400 according to the present general inventive concept includes a back support member 410 having a first surface 410a (i.e., a front surface) and a second surface 410b (i.e., an opposing back surface), an integrated thermal pack compartment 420 formed between the first and second surface 410a and 410b, and first and second adjustable strap members 432 and 434 attached to the back support member 410.

The back support member 410 may include a thermal pack storage compartment 420 configured to receive a thermal pack when in use and also to flip inside out in order to form a strap storage compartment configured to receive and store the plurality of adjustable strap members 432 and 434, when not in use. As such, the first and second adjustable strap members 332 and 334 may be disposed with an internal space of the strap storage compartment formed when the back support member 410 is flipped inside out.

In the present example embodiment, the back support member 410 may be formed in a rectangular shape having a first end 410c, a second end 410d, a top end 410e, and a bottom end 410f. The back support member 410 may include a padding material disposed in between the front surface 410a and the back surface 410b to provide support, thermal therapy, and/or comfort to a user's back 20a. The back support member 410 is configured to engage and support a region of the user's back 20a (e.g., lumbar). The padding material may be formed of foam, cotton, or the like. However, the present general inventive concept is not limited thereto. That is, in alternative example embodiments, the back support member 410 may be formed in various shapes and/or sizes and may be manufactured from various types of materials. In addition, the padding material may include a thermal absorbing material which may absorb heat and/or cold to further apply thermal therapy to the user.

In the present example embodiment, the first and second adjustable strap members 432 and 434 may be coupled to the first and second ends 410c and 410d of the back support member 410. In the present example embodiment, the first and second adjustable strap members 432 and 434 may be inserted into the thermal pack storage compartment 420 when the back support member 410 is flipped inside out.

In addition, the back support member 410 may further include a fastening member 472 having a zipper member 472a used to open and close a top end 410e of the back support member 410 in order to secure the first and second adjustable strap members 332 and 334 within the thermal pack compartment 420, when the back support member 410 is flipped inside out.

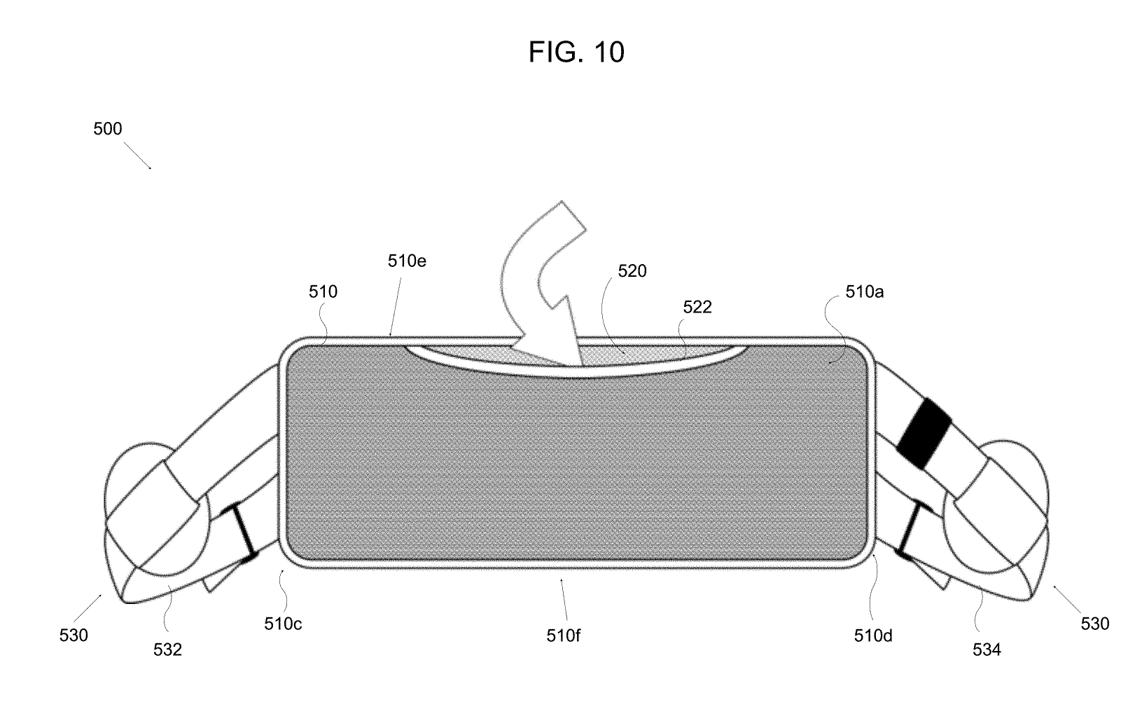

FIG. 10 illustrates a front view of a back support device 500 having an integrated thermal therapy storage compartment according to another example embodiment of the present general inventive concept.

Referring to FIG. 10, the back support device 500 according to the present general inventive concept includes a back support member 510 having a first surface 510a (i.e., a front surface) and a second surface 510b (i.e., an opposing back surface), an integrated thermal pack compartment 520 formed on the front surface 510a of the back support member 510 to store a thermal pack 10 (e.g., an ice pack and/or a heating pad), and a plurality of adjustable strap members 530 attached to the back support member 510. In use, the thermal pack 10 may be stored within the integrated thermal pack storage compartment 520 and the front surface 510a may be placed adjacent to or against the user's back 20 to simultaneously provide back support and thermal therapy to the user 20. In addition, the back support member 510 may be configured to receive and store the plurality of adjustable strap members 532 and 534.

In the present example embodiment, the thermal therapy storage compartment 520 may be formed as a pocket or sleeve 522 which may be formed on the top end 510e of the back support member 510. The sleeve 522 may be formed from an elastic material to support and securely hold a thermal pack 10 disposed within the thermal therapy storage compartment 520.

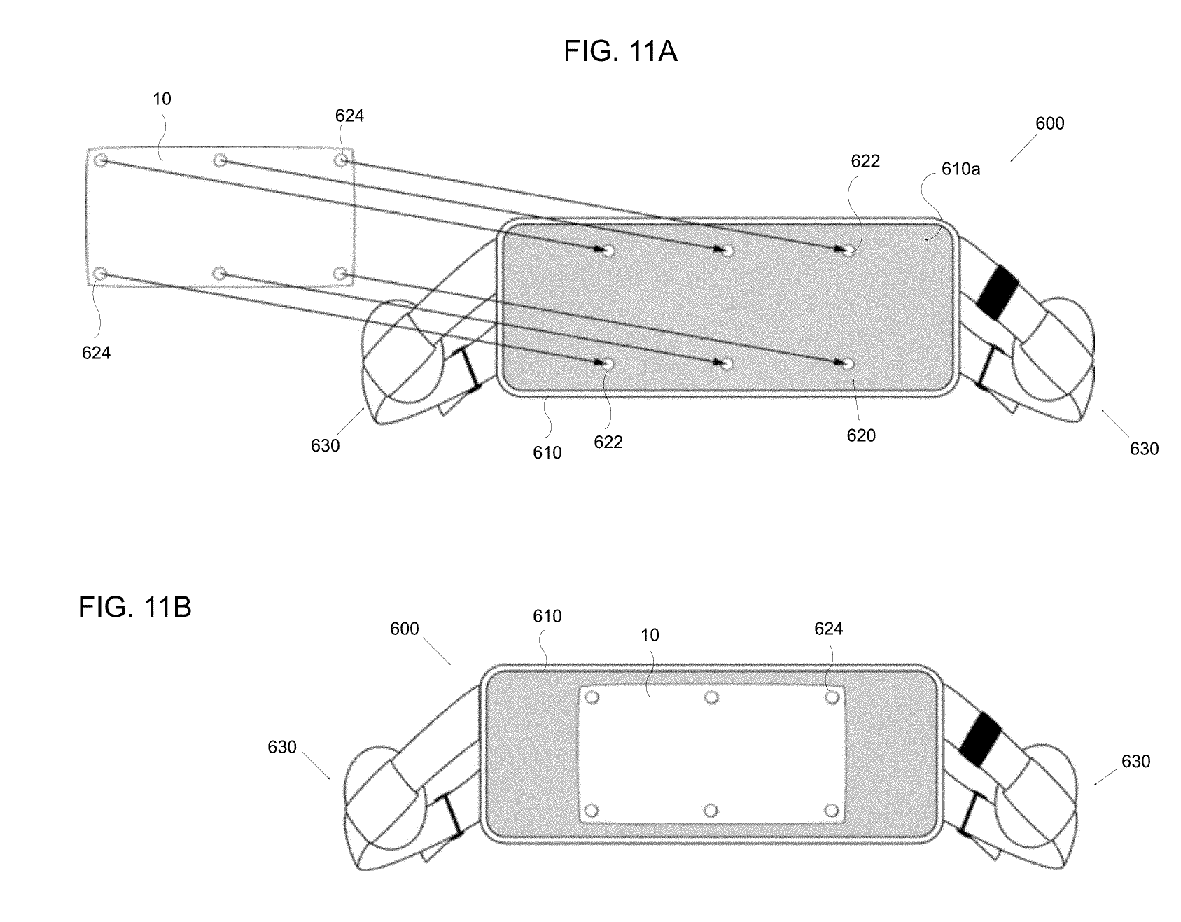

FIG. 11A-11B illustrates a thermal pack 10 and a front view of a back support device 600 according to another example embodiment of the present general inventive concept.

Referring FIG. 11A, the back support device 600 according to the present embodiment is similar to the previous example embodiments. In the present example embodiment, the back support device 600 includes a back support member 610 having a first surface 610a (i.e., a front surface) and a second surface 610b (i.e., an opposing back surface), a thermal pack attachment member 620 integrally formed within the back support member 610 to securely fasten a thermal pack 10 (e.g., an ice pack and/or a heating pad) to the first surface 610a of the back support member 610, and a plurality of adjustable strap members 630 attached to the back support member 610 to help support the user in a proper healthy posture, while sitting. In the present example embodiment, the integrated thermal pack attachment member 620 may include a plurality of magnets 622 coupled and attached to the first surface 610a of the back support member 610 which correspond to a plurality of magnetic surfaces 624 coupled to the thermal pack 10. As such, the thermal pack 10 may be detachably coupled to the first surface 610a of the back support member 610.

Referring to FIG. 11B, the thermal pack 10 is coupled to the first surface 610a of the back support member 610 when one or more of the plurality of magnetic surfaces 624 coupled to the thermal pack 10 contact one or more of the plurality of magnets 622 of the back support member 610. However, the present general inventive concept is not limited thereto. That is, in alternative example embodiments, the integrated thermal pack attachment member 620 may include various types of fastening means including conventional mechanical fasteners such as snaps, buttons, clips, or the like.

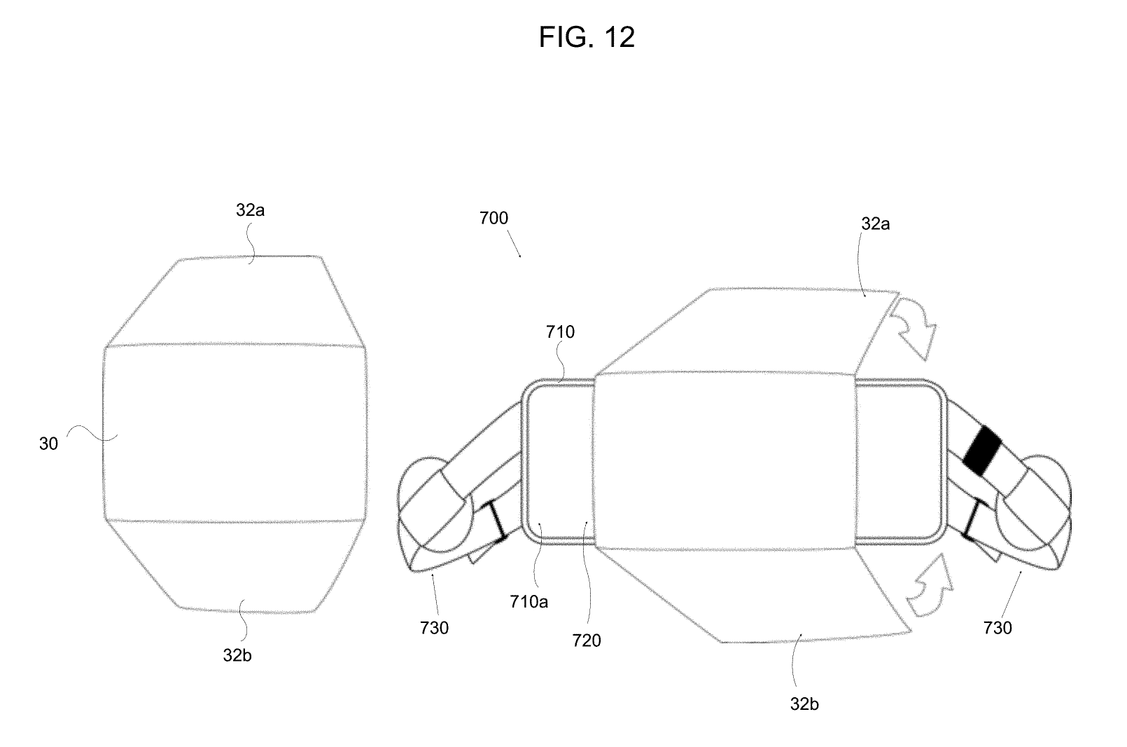

FIG. 12 illustrates a wrap-around thermal pack 30 according to an example embodiment of the present general inventive concept.

Referring FIG. 12, the back support device 700 according to the present embodiment is similar to the previous example embodiments. In the present example embodiment, the back support device 700 includes a back support member 710 having a first surface 710a (i.e., a front surface) and a second surface 710b (i.e., an opposing back surface), a thermal pack attachment member region 720 integrally formed within the back support member 710 to receive a specifically designed wrap around thermal pack 30 (e.g., an ice pack and/or a heating pad) to be secured around the first surface 710a of the back support member 710, and a plurality of adjustable strap members 730 attached to the back support member 710 to help support the user in a proper healthy posture, while sitting.

In the present example embodiment, the wrap around thermal pack 30 includes first and second attachment members 32a and 32b which are attached to each other after being wrapped around the back support member 710. As such, the thermal pack 30 may be detachably coupled to the first surface 710a of the back support member 710. The first and second attachment members 32a and 32b may be attached to each other by various fasteners including velcro, snaps, buttons, clips, magnets, or the like. However, the present general inventive concept is not limited thereto.

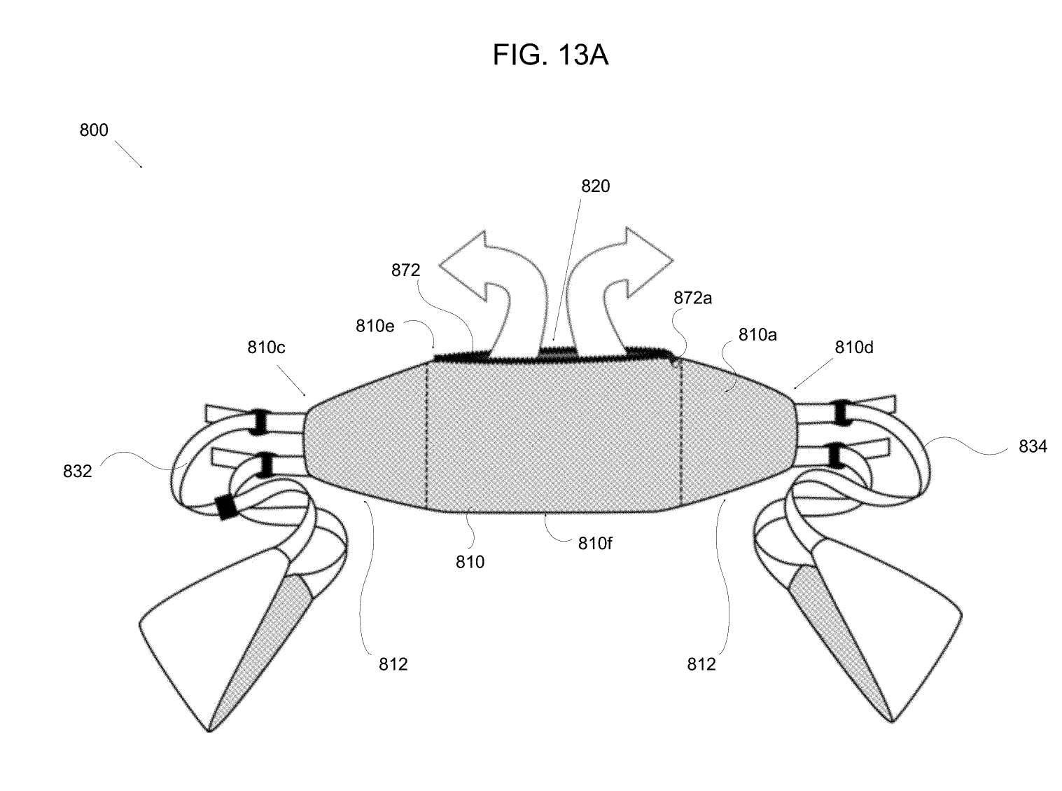

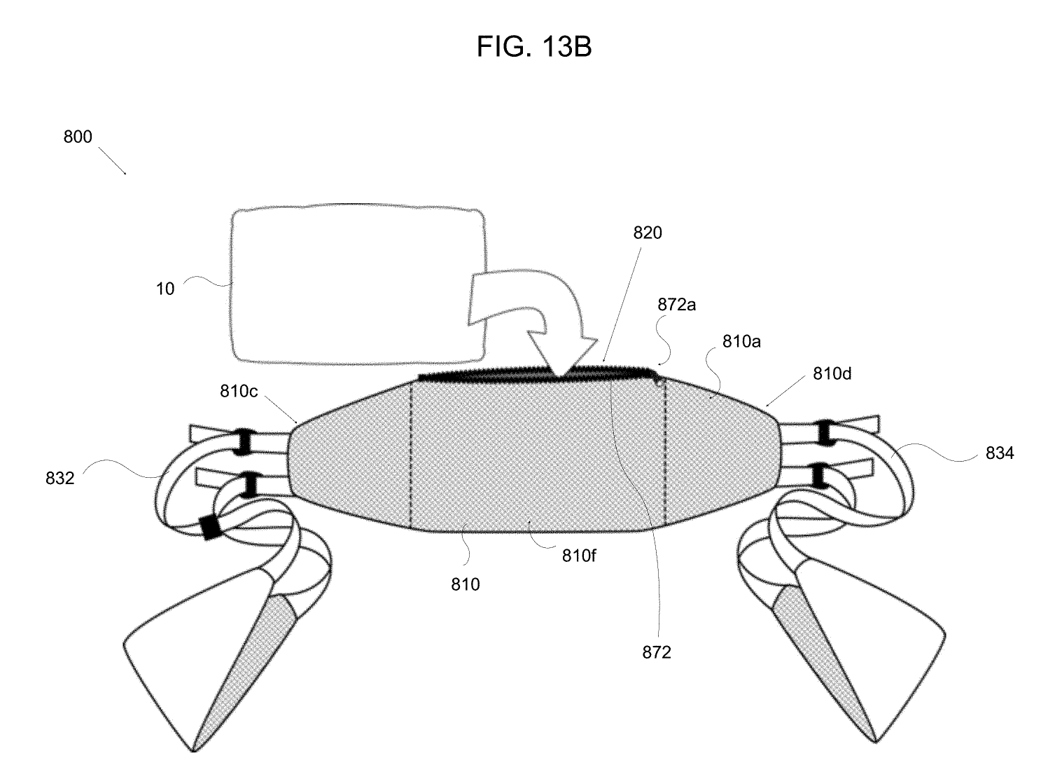

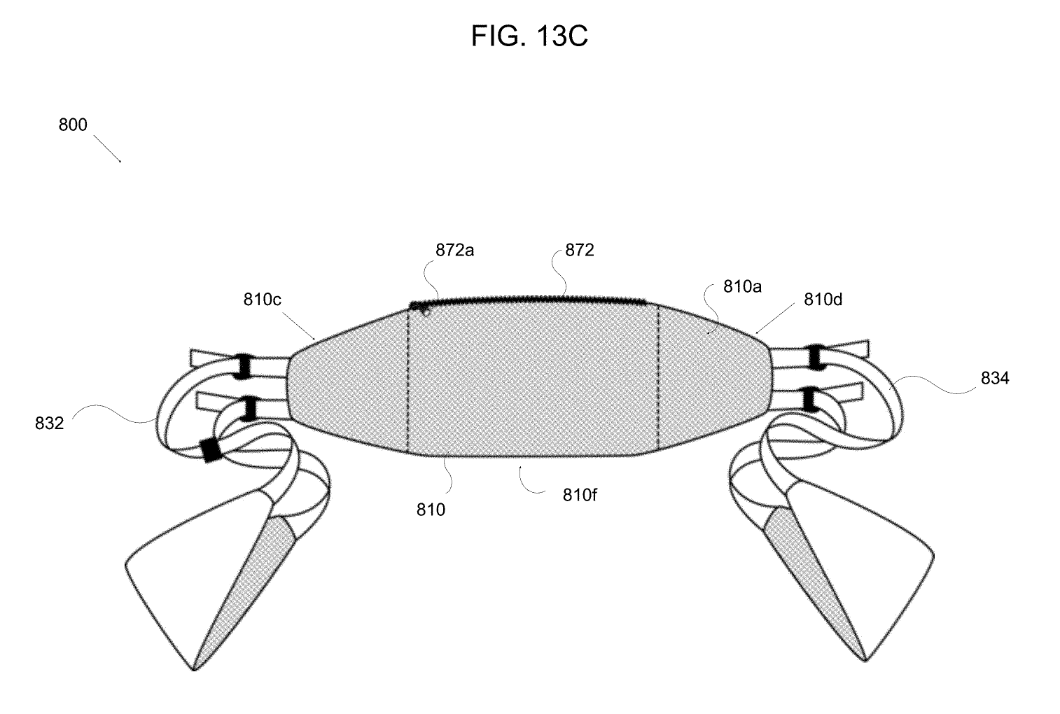

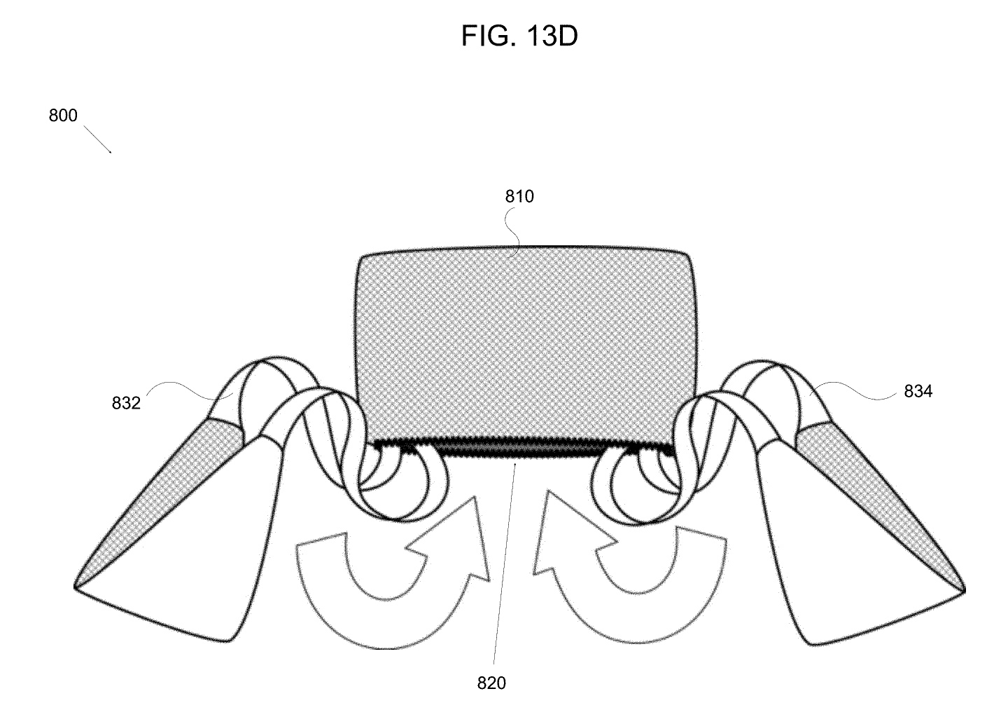

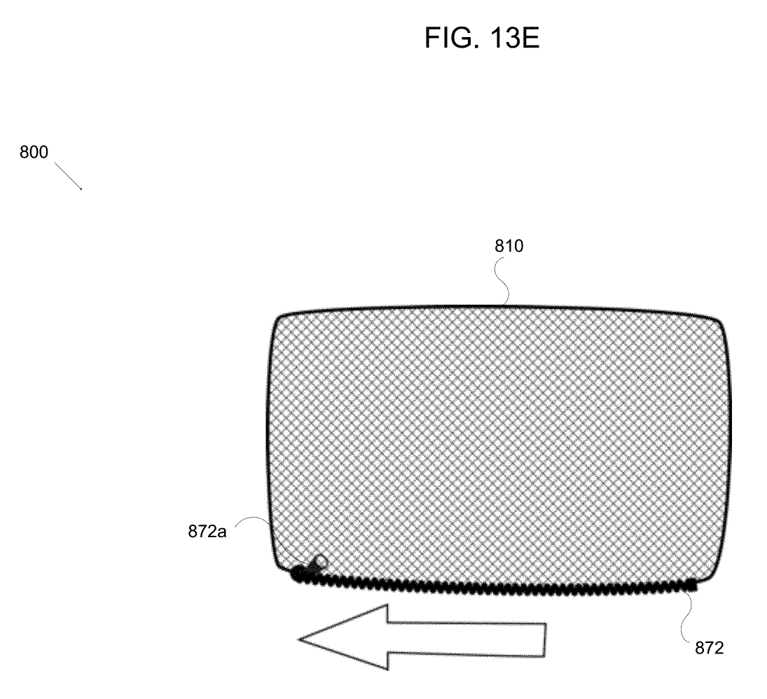

FIGS. 13A-13E illustrate a back support device 800 and method of storing the adjustable strap members in the back support device 800 according to another example embodiment of the present general inventive concept.

Referring to FIGS. 13A-13E, the back support device 800 according to the present general inventive concept includes a back support member 810 having a first surface 810a (i.e., a front surface) and a second surface 810b (i.e., an opposing back surface), an integrated thermal pack storage compartment 820 formed between the first and second surface 810a and 810b, and first and second adjustable strap members 832 and 834 attached to the back support member 810. In alternative example embodiments, the first and second adjustable strap members 832 and 834 may further include knee pads as described in previous example embodiments.

The back support member 810 may include a thermal pack storage compartment 820 configured to receive a thermal pack 10 when in use and also to flip inside out in order to form a strap storage compartment configured to receive and store the plurality of adjustable strap members 832 and 834, when not in use.

In the present example embodiment, the back support member 810 may be formed in a rectangular shape having tapered end portions 812 and having a first end 810c, a second end 810d, a top end 810e, and a bottom end 810f. The back support member 810 may include a padding material disposed in between the front surface 810a and the back surface 810b to provide support, thermal therapy, and/or comfort to a user's back 20a. The back support member 810 is configured to engage and support a region of the user's back 20a (e.g., lumbar). The padding material may be formed of foam, cotton, or the like. However, the present general inventive concept is not limited thereto. That is, in alternative example embodiments, the back support member 810 may be formed in various shapes and/or sizes and may be manufactured from various types of materials. In addition, the padding material may include a thermal absorbing material which may absorb heat and/or cold to further apply thermal therapy to the user.

In the present example embodiment, the first and second adjustable strap members 832 and 834 may be coupled to the first and second ends 810c and 810d of the back support member 810. In the present example embodiment, the first and second adjustable strap members 832 and 834 may be inserted into a strap storage compartment formed as the back support member 810 is flipped inside out through the thermal pack storage compartment 820.

In addition, the back support member 810 may further include a fastening member 872 having a zipper member 872a used to open and close a top end 810e of the back support member 810 in order to secure the first and second adjustable strap members 832 and 834 within the thermal pack compartment 820, when the back support member 810 is flipped inside out.

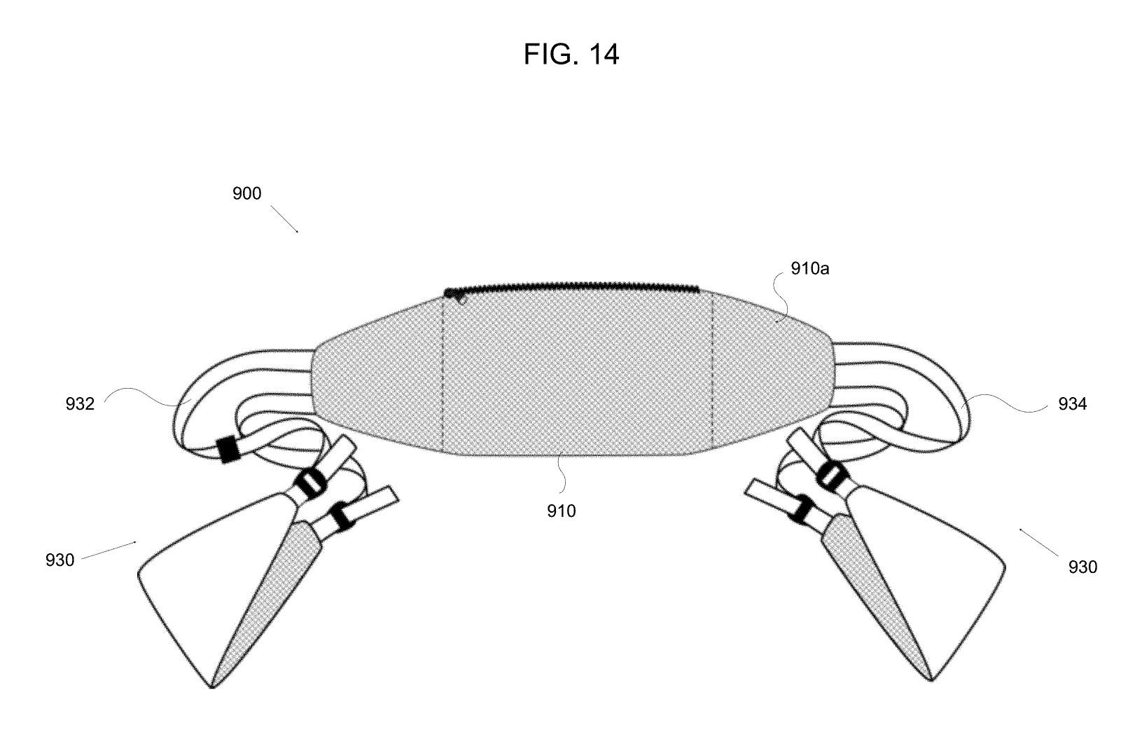

FIG. 14 illustrates a back support device 900 according to another example embodiment of the present general inventive concept.

Referring FIG. 14, the back support device 900 according to the present embodiment is similar to the previous example embodiments. In the present example embodiment, the back support device 900 includes a back support member 910 having a first surface 910a (i.e., a front surface) and a second surface 910b (i.e., an opposing back surface), a thermal pack attachment compartment 920 integrally formed within the back support member 910 to store a thermal pack 10 (e.g., an ice pack and/or a heating pad), and a plurality of adjustable strap members 930 attached to the back support member 910 to help support the user in a proper healthy posture, while sitting.

The present general inventive concept provides a combined back support and thermal therapy apparatus that provides thermal therapy to the user, while at the same time using the user's knees to help support the user in a proper healthy posture, when sitting. The combined back support and thermal therapy apparatus may be flipped inside out to thereby form a storage pouch for the entire apparatus for easy storage and transport.

Although a few exemplary embodiments of the present general inventive concept have been illustrated and described, it will be appreciated by those skilled in the art that changes may be made in these exemplary embodiments without departing from the principles and spirit of the general inventive concept, the scope of which is defined in the appended claims and their equivalents.

What is claimed is:

1. A combined back support and thermal therapy apparatus for supporting and applying thermal therapy to a user's back, the apparatus comprising:
a back support member having a first surface and an opposing second surface, the first surface configured to face and support a user's back;
a thermal therapy storage compartment integrally formed between the first and second surfaces of the back support member and configured to store a thermal pack; and
first and second adjustable strap members coupled to opposite ends of the back support member, each of the first and second adjustable strap members are configured to wrap around a user's knee,
wherein the back support member folds along a center region to form a strap storage compartment defined within the second surface, the strap storage compartment configured to receive and store the first and second adjustable strap members.

2. The apparatus of claim 1, further comprising a tension application member coupled at least one of the first adjustable strap member and the second adjustable strap member.

3. The apparatus of claim 2, wherein the tension application member is configured to allow the user to adjust and fix a length of the adjustable strap member.

4. The apparatus of claim 3, wherein the user's knees applies a force to the back support member through the first and second adjustable strap members to support the user in a proper posture position.

5. The apparatus of claim 4, further comprising knee pads coupled to the first and second adjustable strap members, the knee pads configured to distribute a force over a surface of the user's knees.

6. The apparatus of claim 5, further comprising a thermal therapy storage compartment integrally formed with the knee pads and configured to store a thermal pack to provide thermal therapy to the user's knees.

7. The apparatus of claim 4, wherein the knee pads are formed in a wedge shape to evenly distribute a force over a surface of the user's knees.

8. The apparatus of claim 4, further comprising a sleeve formed on the first surface of the back support member configured to provide access to the thermal therapy storage compartment.

9. The apparatus of claim 1, further comprising a fastening member coupled to a perimeter of the back support member to open and close the strap storage compartment from an external environment.

10. The apparatus of claim 8, wherein the fastening member includes one of a snap, a button, a zipper, a velcro™ and a clip.

11. The apparatus of claim 1, further comprising a clip member configured to detachably connect the first adjustable strap member to the second adjustable strap member to prevent the user's knees from spreading apart.

12. An integrated storage pouch apparatus for a back support and thermal therapy device for supporting and applying thermal therapy to a user's back, the apparatus comprising:
a back support member having a first surface and an opposing second surface, the first surface configured to face and support a user's back;a thermal therapy storage compartment integrally formed between the first and second surfaces of the back support member and configured to store a thermal pack; and
first and second adjustable strap members coupled to opposite ends of the back support member, each of the first and second adjustable strap members are configured to wrap around a user's knee,
wherein the back support member folds inside out to through the thermal therapy storage compartment to form a strap storage compartment configured to receive and store the first and second adjustable strap members.

13. The apparatus of claim 12, further comprising a tension application member coupled to at least one of the first adjustable strap member and the second adjustable strap member to adjust a length of the adjustable strap member.

14. The apparatus of claim 13, wherein the user's knees applies a force to the back support member through first and second adjustable straps to support the user in a proper posture position.

15. The apparatus of claim 14, further comprising knee pads coupled to the first and second adjustable strap members, the knee pads configured to distribute a force over a surface of the user's knees.

16. The apparatus of claim 15, wherein the knee pads are formed in a wedge shape to evenly distribute a force over a surface of the user's knees.

17. The apparatus of claim 16, further comprising a thermal therapy storage compartment integrally formed with the knee pads and configured to store a thermal pack to provide thermal therapy to the user's knees.

18. The apparatus of claim 12, further comprising a fastening member coupled to a top end of the back support member to open and close the thermal therapy storage compartment from an external environment.

19. The apparatus of claim 12, wherein the fastening member is configured to open and close the thermal therapy storage compartment and the strap storage compartment when the back support member is flipped inside out.

20. The apparatus of claim 12, further comprising a clip member configured to detachably connect the first adjustable strap member to the second adjustable strap member to prevent the user's knees from spreading apart.
